(19) United States
(12) Patent Application Publication
(10) Pub. No.: US 2010/0262092 A1
(43) Pub. Date: Oct. 14, 2010

(54) DRESSING

(75) Inventor: Edward Yerbury Hartwell, York (GB)

Correspondence Address: KNOBBE MARTENS OLSON & BEAR LLP, 2040 MAIN STREET, FOURTEENTH FLOOR, IRVINE, CA, 92614, US

(73) Assignee: Smith & NEphew Plc, Londpn (GB)

(21) Appl. No.: 12/746,504

(22) PCT Filed: Nov. 26, 2008
(86) PCT No.: PCT/GB08/51114

§ 371 Date: Jun. 4, 2010

(30) Foreign Application Priority Data
Dec. 6, 2007 (GB) 0723874.4

(51) Int. Cl. A61F 13/00

(52) U.S. Cl. 604/304

(57) ABSTRACT

A dressing for the application of topical negative and/or positive pressure therapy to a wound is described together with a method for creating the dressing, the dressing comprising in use: an optional layer of a pressure resistant porous material adjacent a surface of a wound to be treated; a first layer of a flexible wound covering and sealing material on top of the optional pressure resistant porous material adapted, in use, to surround the wound and seal against sound tissue to form, in use, a first sealed cavity with the wound; a first conduit having a first end adapted to communicate with an interface between said optional layer of porous material and said first layer of flexible wound covering and sealing material and a second end adapted to communicate with vacuum means to establish a negative pressure, in use, between said first covering and sealing material layer and a wound surface; a resiliently compressible wound packing material on top of said first layer of covering and sealing material; a second conduit having a first end adjacent said resiliently compressible wound packing material and a second end adapted to communicate with positive or negative pressure generating means; and a second layer of flexible covering and sealing material over said resiliently compressible wound packing material to form, in use, a second sealed cavity above said first sealed cavity and said wound.

The present invention relates to a dressing and a method a making a dressing particularly, though not exclusively, for use in topical negative pressure (TNP) therapy of mammals.

TNP therapy often involves the provision within a dressing to which a negative or a positive pressure is applied a bag member (sometimes also referred to as a bladder) which may be used to at least partially fill a volume of a wound, for example. Furthermore, fluctuating pressure or pressure cycling may be applied to the bag member in order to work the tissue in and around the wound region for therapeutic reasons, for example.

There are many commercially available bag members which may be included in a wound dressing, however, commercially available bag members suffer from the disadvantage that they are obviously of predetermined size and shape, for example, and there may not be available a bag member of a corresponding form to that of the wound which needs treatment. Such unavailability may not be just the unavailability in the particular hospital formulary or store, for example, but a suitably sized and shaped bag member may not be available at all commercially.

In our co-pending International patent application, WO 2004/037334, apparatus, a wound dressing and a method for aspirating, irrigating and cleansing wounds are described. In very general terms, this invention describes the treatment of a wound by the application of topical negative pressure (TNP) therapy for aspirating the wound together with the further provision of additional fluid for irrigating and/or cleansing the wound, which fluid, comprising both wound exudates and irrigation fluid, is then drawn off by the aspiration means and circulated through means for separating the beneficial materials therein from deleterious materials. The materials which are beneficial to wound healing are recirculated through the wound dressing and those materials deleterious to wound healing are discarded to a waste collection bag or vessel.

In our co-pending International patent application, WO 2005/04670, apparatus, a wound dressing and a method for cleansing a wound using aspiration, irrigation and cleansing wounds are described. Again, in very general terms, the invention described in this document utilises similar apparatus to that in WO 2004/037334 with regard to the aspiration, irrigation and cleansing of the wound, however, it further includes the important additional step of providing heating means to control the temperature of that beneficial material being returned to the wound site/dressing so that it is at an optimum temperature, for example, to have the most efficacious therapeutic effect on the wound.

In our co-pending International patent application, WO 2005/105180, apparatus and a method for the aspiration, irrigation and/or cleansing of wounds are described. Again, in very general terms, this document describes similar apparatus to the two previously mentioned documents hereinabove but with the additional step of providing means for the supply and application of physiologically active agents to the wound site/dressing to promote wound healing.

The content of the above references is included herein by reference.

All of the wounds to which the above documents are addressed may require the provision of a bag member or bladder within a wound dressing and those currently available may be unsuitable. Indeed, our co-pending International patent application WO 2004/037334 referred to above shows at FIGS. 13A and 20 embodiments employing pre-formed inflatable bladders used in apparatus for wound therapy.

The present invention is intended to overcome or mitigate the disadvantages of the prior art and to provide a method of making a bag member for use in a dressing to which positive or negative pressures may be applied.

It is further intended to provide a method of making a suitably sized and shaped bag member in a dressing for a particular wound from materials commonly available in most hospital formularies or stores.

According to a first aspect of the present invention there is provided a dressing for the application of topical negative and/or positive pressure therapy to a wound, the dressing comprising in use:
an optional layer of a pressure resistant porous material adjacent a surface of a wound to be treated;
a first layer of a flexible wound covering and sealing material on top of the optional pressure resistant porous material adapted, in use, to surround the wound and seal against sound tissue to form, in use, a first sealed cavity with the wound;
a first conduit having a first end adapted to communicate with an interface between said optional layer of porous material and said first layer of flexible wound covering and sealing material and a second end adapted to communicate with vacuum means to establish a negative pressure, in use, between said first covering and sealing material layer and a wound surface;
a resiliently compressible wound packing material on top of said first layer of covering and sealing material;
a second conduit having a first end adjacent said resiliently compressible wound packing material and a second end adapted to communicate with positive or negative pressure generating means; and
a second layer of flexible covering and sealing material over said resiliently compressible wound packing material to form, in use, a second sealed cavity above said first sealed cavity and said wound.

According to a second aspect of the present invention there is provided a method of providing a dressing including a bag member on a wound on a mammal, the method comprising the steps of:
optionally placing a layer of a pressure resistant material which allows fluid transmission therethrough on a bed of the wound;
placing an end of a first conduit adjacent said optional layer of pressure resistant material;
adhering a first layer of a flexible, wound covering and sealing material on top of the aspirant conduit and layer of pressure resistant material such that said first layer is sealed to skin surrounding the wound and to said first conduit so as to form a first sealed cavity over said wound;
placing a resiliently compressible wound packing material in the wound cavity on top of said first sealing layer material; placing an end of a second conduit adjacent said wound packing material; and,
placing a second layer of a flexible, adhesive coated wound covering and sealing material over said wound packing material and an area surrounding said wound to seal thereagainst and to said second conduit so as to form a second sealed cavity over said wound.

The layer of pressure resistant material is optional since it may not be needed in the case of small wounds since the vacuum within the first sealed cavity of the dressing may be able to reach all parts of the wound bed without the pressure resistant layer.

It is preferred that the first and second flexible, wound covering and sealing materials are adhesive coated, semi-permeable materials allowing the wound/dressing to breathe. Suitably such materials are thin film materials commonly available and may be made from polyurethanes, such as polyester and polyether polyurethanes, elastomeric polyether polyesters and the like, for example. Common, commercially available materials include OPSITE (trade mark) and TEGADERM (trade mark), for example. The use of semi-permeable materials is primarily to allow the wound region to breath and prevent maceration of the wound periphery.

In the present invention the dressing is effectively sealed to the skin surrounding the wound by means of the flexible, adhesive coated film material. However, the term “sealed” is not an absolute requirement nor practically attainable since many flexible dressing membrane materials forming the wound cover are composed of semi-permeable plastics materials which are well known to those skilled in the art. The term semi-permeable is defined as being permeable to water vapour and gases but not liquids and has a transmissibility of moisture vapour greater than 500 g/sq.m/per 24 hr period; if the transmission of moisture vapour is less than this figure then the material is not considered to be semi-permeable. Furthermore, there is almost inevitably some leakage between the skin to which the sealing dressing material is adhered, usually by well known pressure sensitive adhesives, due to hairs and/or other skin surface irregularities and/or imperfections which are not easily completely sealed in absolute terms. Examples of the types of self adhesive, flexible dressing drape materials which are ordinarily used in TNP type therapy as sealing membranes over and around wounds are listed hereinabove and are well known to those skilled in the art and will not be elaborated on further herein unless necessary.

A particular advantage of the type of bag dressing according to the present invention in comparison with conventional porous type wound packing materials is that the fluids around the wound may be rapidly aspirated away from the wound thus reducing build up of toxins and bacterial burden. Furthermore, pressure in the bag (second sealed cavity) may be changed independently of suction applied to the wound (first) cavity and tissue thus allowing for pain reduction and allowing the application of varying mechanical stress to the tissue.

The layer of pressure resistant material which allows fluid transmission therethrough placed on the wound bed is intended to both support the overlying first flexible layer of sealing material and to prevent it being drawn down into contact with the wound bed when a negative pressure (relative to atmospheric pressure) is applied via the first conduit to the first sealed cavity over the wound. Thus, the purpose of the pressure resistant layer in contact with the wound bed is to resist being crushed by the negative pressure level employed and to permit the establishment of a uniform negative pressure distribution over the whole area of the wound bed and thus render the entire wound area available and susceptible to the benefit of the reduced pressure so as to stimulate blood flow thereto and to all of the remaining known benefits of TNP therapy.

A further advantage of the present invention is that the optional pressure resistant material which contacts the wound may be kept thin and consequently drapeable such that it is able to conform to and remain in contact with the wound surface.

The pressure resistant layer may be porous or may have surface texture or topography such as channels or indentations which allow the transfer of fluids such as aspirant fluid or wound exudate over the area of the layer. The layer of pressure resistant material may be bio-compatible and/or bio-absorbable such as collagen, oxidised cellulose, chitosan, INTEGRA (TRADE NAME) or other suitable materials know to those skilled in the art, for example. In such materials it may be advantageous to have pores optimised for promoting the growth of tissue.

It is preferred that the pressure resistant material layer is non-adherent to raw tissue and has small pores sufficiently small to prevent tissue growth into the pores. Such materials are often referred to as “wound contact layers”. Whilst the material is referred to as pressure resistant this is only in so far as the material is required to maintain adequate porosity at the desired maximum extent of negative pressure which may be around 250 mmHg below atmospheric pressure.

The pressure resistant material may be bio-absorbable.

The first sealed cavity may also have an optional thin layer of wound packing material on top of the pressure resistant material layer. The layer of wound packing material over the wound may not be required in all or even most cases but may be valuable in some large wounds or tunnelling or fistula type wounds.

The first conduit may be a flexible plastics material tube having an array of holes therein in the wall thereof in the portion of the conduit which is contained within the dressing first sealed cavity. The first conduit is to apply a negative pressure to the wound first sealed cavity of the dressing thereby both aspirating the wound surface and inter alia stimulating blood flow thereto and also removing wound exudates from the wound site so removing materials which may be detrimental to wound healing. The first conduit may be in operable connection to apparatus having means such as vacuum pump means, for example, for applying a negative pressure.

In some embodiments of the present invention, optionally one or more additional conduits may also be sealed into operable connection with the first sealed cavity, these conduits being for, for example, the supply of irrigation or cleansing fluids; pharmaceutical agents intended to have healing therapeutic effects on the wound; or to return beneficial fluids which have been removed from the wound site as exudates and mixed with other fluids and treated by, for example, a dialysis technique to the wound. Such techniques are fully discussed in our co-pending International patent applications referenced hereinabove.

Thus, the first sealed cavity provided by the first sealing layer of flexible, adhesive coated film material may render all of the known beneficial effects of TNP therapy and those additional known techniques mentioned hereinabove in relation to the International patent applications of common ownership herewith to the wound. The first sealed cavity provides in effect an isolated, sealed therapeutic environment for wound healing.

Before carrying out the remaining steps of the method according to the present invention it may be beneficial to employ an optional additional step of applying a negative pressure to the first sealed cavity in order to draw the constituent layers down towards the wound.

The resiliently compressible wound packing material is then placed on top of the first sealing layer such that, preferably, it is just above skin level and is shaped so as to conform generally to the wound shape.

One purpose of the resiliently compressible packing material is to prevent opposite edges of the wound from growing together too quickly and overgrowing the wound itself to form a closed wound cavity.

Preferably, the resiliently compressible packing material may be transparent to enable a clinician, for example, to view the wound to assess healing progress. An example of a suitable material may be a polyurethane based plastics foam material.

The resiliently compressible wound filler may be any suitable porous material such as foam, mesh material, knitted material, corrugated material, for example, and relatively very large pore sizes up to about 10 mm may be employed. Larger pore sizes may be used but there is a limit imposed where the adhesive film may be pulled into the pores by negative pressure effect and may stick to the porous material. However, this latter problem may be overcome by interposing a sheet of a suitable material between the resiliently compressible wound packing material and the second flexible wound covering and sealing material. Suitable materials may include polyethylene and polyvinyl acetate, for example.

The second conduit may be placed adjacent the resiliently compressible wound packing material and the second layer of flexible, adhesive coated sealing material put in place to create the second sealed cavity. The second sealed cavity is isolated from the wound and is present to work or stress the wound tissue for its beneficial effect thereon. The second conduit may be connected to suitable pump means to apply a positive pressure (relative to atmospheric) and/or a negative pressure to the second sealed cavity. In most of the prior art which shows bag members or bladders, they are employed to work or stress the tissue in the wound region by inflation thereof and cycling and/or pulsing, for example, of the pressure within the bladder in a number of different ways. However, none of the bladders shown in the prior art possess the resiliently compressible wound filler of the present invention. Thus the wound being treated by the dressing created by the method of the present invention may be worked or stressed by providing a positive pressure in the second sealed cavity and/or a negative pressure therein. When a negative pressure is applied the effect is that ambient atmospheric pressure serves to compress the resiliently compressible wound filler material which itself, being resilient provides a positive force on the wound bed and surrounding tissue due to its compression and being held against the wound by ambient atmospheric pressure. In the dressing according to the present invention the ambient atmosphere is not applied to the underside of the second sealed cavity as this space is occupied by the first sealed cavity which itself is sealed from ambient atmospheric pressure on the wound side thus, the stresses acting to expand the wound filler material when compressed act in a downwardly direction against the wound region. The forces provided by the compressed wound filler on the wound may be controlled by, for example, the degree of porosity, pore size, the material from which it is composed and the level of positive or negative pressure applied to the second sealed cavity. Thus, the dressing of the present invention is able to work or stress the wound over a much greater range of conditions. The range of pressures over which the second sealed cavity may be used is from +300 mmHg to −400 mmHg. Typical pressures may lie in the range from about +50 to −200 mmHg, more preferably from +25 mmHg to −150 mm. Inclusion of the compressible filler gives greater range and control particularly for contraction of the wound.

The resiliently compressible wound filling material may be a foam formed in situ in the wound according to our co-pending patent application, PCT/GB2008/050268, the content of which is incorporated herein by reference.

The fluid used to apply pressure to the second sealed cavity is preferably a gas such as air, for example, but could be a liquid such as water. However, a gas is preferred owing to its compressible nature in case a patient rolls on top of the dressing causing discomfort. The fluid may be pressure pulsed to “work” the tissue surface.

The fluid used to apply pressure may be temperature controlled.

The second layer of flexible, adhesive coated sealing material which finally seals the wound and second cavity may be formed from a stronger film material or a reinforced material so as to resist upwardly directed stresses which put the material under tension, which may cause “ballooning” when the second cavity is inflated with a positive pressure, for example.

Additional conduits may be placed in the first and/or second sealed cavities in order to monitor the pressures therein and/or to supply medication thereto.

A particular advantage of the method of making a dressing according to the present invention is that all of the component parts thereof may be tailored to fit the wound under consideration and that no compromises are required thus providing improved wound therapy.

The first and second conduits may be combined into a single, multi-lumen conduit. In this case the first covering and sealing layer may be provided with a port member which accepts the multi-lumen conduit and the fluid flow paths in the multi-lumen conduit being directed as appropriate by the port into the respective first and second sealed cavities.

Such port members may also allow the pressure, negative or positive, in the first and/or second sealed cavities to be monitored and/or controlled by means of additional lumens connected to control/monitoring devices remote from the wound. Such devices may comprise the vacuum/pressure generating means and transducer means, for example, to monitor the pressure at the wound. Such transducer means may be further linked to control means to control the pressure/vacuum generating means and/or valve/air bleed means into the first and/or second sealed cavities.

According to a third aspect of the present invention there is provided a port member for a dressing, the port member comprising a body portion having flow passages adapted to co-operate with at least two lumens; a face portion adapted to be adhered to a flexible membrane material; and, the flow passages being directed on either side of said face portion.

The port member has at least one fluid flow passage configured to communicate with a region on a first side of the face portion.

The port member has at least one fluid flow passage configured to communicate with a region on a second side of the face portion.

A suitable port member may be moulded from a soft plastics material such as polyurethane, silicone or polypropylene, for example, and comprises a base flange face portion having an adhesive layer on at least a peripheral portion of the base flange to allow the port member to be adhered or welded to the first flexible covering and sealing material layer of the dressing according to the present invention. The port member may have at least two fluid flow passages therein adapted to co-operate with first and second conduit lumens. When the port member is adhered to the first flexible covering and sealing material the fluid flow passages in the port member permit fluid flow on each side of the first flexible covering and sealing material film. Thus, one lumen in the conduit is able to apply a negative pressure to the first sealed cavity and the second lumen is able to apply either a negative pressure or a positive pressure to the second sealed cavity as appropriate.

The adhesive layer of the base flange face portion may be protected before use with a known peelable and discardable paper such as siliconised paper for example, prior to adhering the port member to a dressing covering and sealing material.

According to a fourth aspect of the present invention there is provided a kit for the provision of a topical negative pressure therapy dressing for a wound, the kit comprising: pressure resistant porous material for placement, in use, adjacent a wound surface; flexible covering and sealing material adapted, in use, for adhering to sound skin; resiliently compressible porous wound packing material; a conduit comprising at least two lumens; and, a port member having at least two flow passages adapted to co-operate with said two lumens.

In order that the present invention may be more fully understood examples will now be described by way of illustration only with reference to the accompanying drawings, of which:

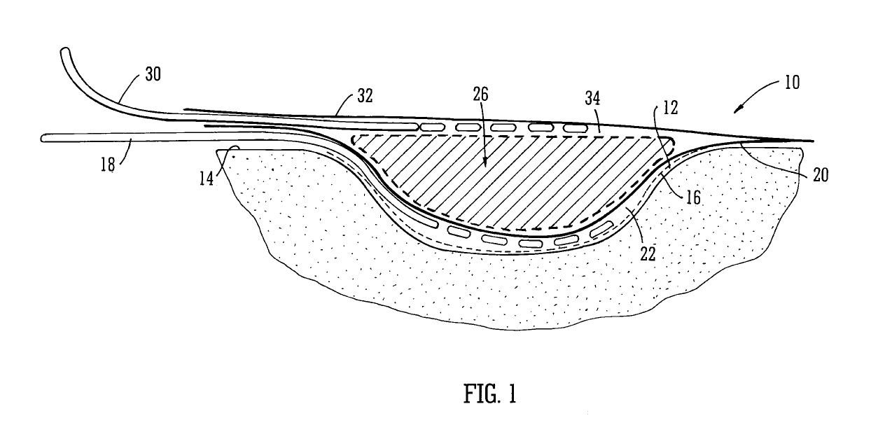

FIG. 1 shows a cross section through a wound having a dressing according to a first embodiment made by the method according to the present invention;

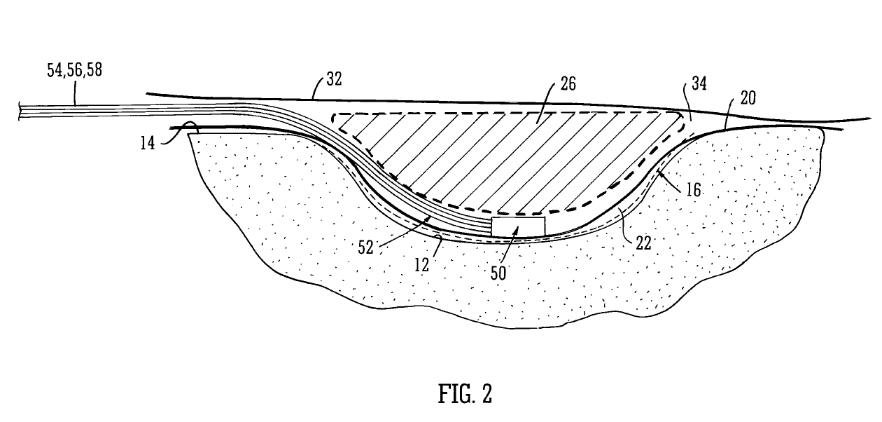

FIG. 2 shows a similar cross section to FIG. 1 but of a second embodiment; and

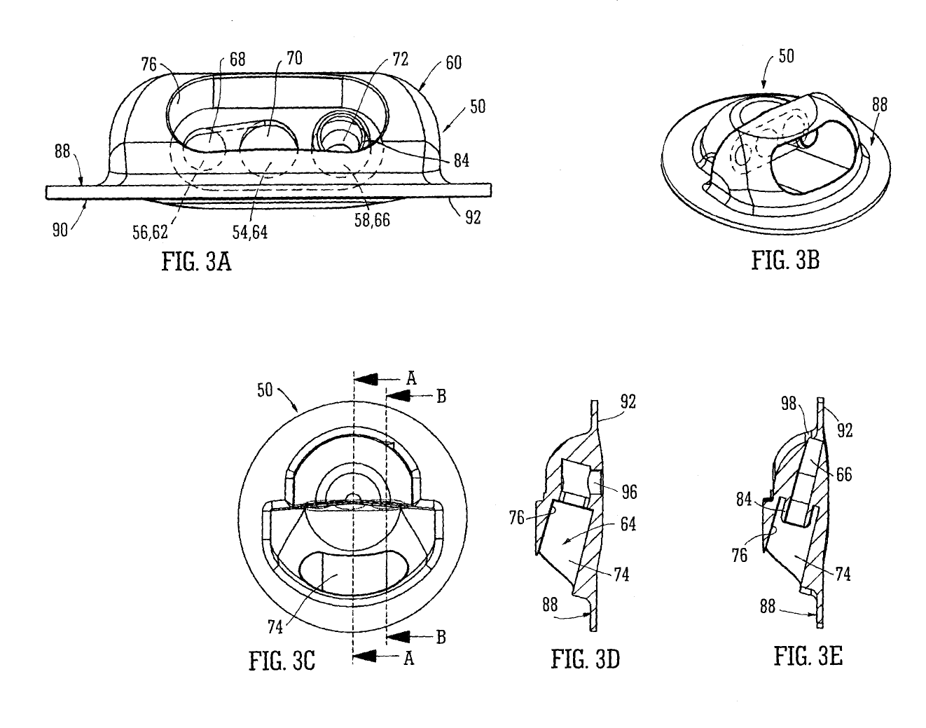

FIGS. 3A to 3E show various views of a port member for use with the dressing shown in the second embodiment of FIG. 2 with FIG. 3A showing a front elevation; FIG. 3B showing a perspective view of the port member; FIG. 3C showing a plan view of the port member from below; and FIGS. 3D and 3E being cross sections through lines A-A and B-B of FIG. 3C, respectively.

Referring now to FIG. 1 and where a wound having a dressing is denoted generally at 10. The wound is denoted at 12 in the form of a deep depression in the tissue 14. The dressing is made by first placing a layer of wound contact material 16 in direct contact with the wound bed. The wound contact material 16 is a porous, pressure resistant material which resists crushing at negative pressures of a maximum of about −250 mmHg below atmospheric and serves to maintain a uniform pressure distribution over the area of the wound. Suitable materials may include Gazetex (trade mark) gauze bandage roll supplied by Derma Sciences Inc., CAVICARE (trade mark) supplied by Smith & Nephew, open cell reticulated polyurethane foam, Mepitel (trade mark) supplied by Molnlycke, for example. A first conduit 18 is laid in the wound on top of the wound contact layer, the conduit being a soft flexible plastics material able to conform to the wound shape. A first flexible, adhesive coated film drape material 20 is then laid over the whole area of the wound and over a surrounding area of sound tissue to adhere thereto and thus to create a first sealed cavity 22 adjacent the wound 12, the first conduit 18 being sealed to the first cavity by pinching the drape material 20 therearound in known manner (not shown). A piece of resiliently compressible foam wound packing material 26 is placed in the wound 12 on top of the drape material 20, the packing material being shaped to the wound and standing slightly proud of the surrounding sound tissue 14. The first sealed cavity 22 may have a negative pressure applied thereto via the conduit 18 to draw the first drape material 20 down onto the wound contact material 16 so that the wound shape may be more accurately ascertained prior to preparation and placing of the wound packing material 26. A second conduit 30 is then placed on or in the wound packing material 26, the conduit 30 being operably connected to means for providing a positive and/or negative pressure in the final dressing. Lastly a second layer of flexible, adhesive coated film drape material 32 is laid over the entire wound and surrounding sound tissue to bond, preferably with the border of the first layer of sealing material 20 or, to sound tissue, the second drape material being pinched around the conduit 30 to seal therewith (not shown) and to form a second sealed cavity 34.

Referring now to FIGS. 2 and 3 and where FIG. 2 shows a similar cross section to that of FIG. 1 and FIGS. 3A to 3E show details of a port member associated with the dressing according to this second embodiment. In this second embodiment there are: a wound contacting layer 16 of a pressure resistant porous material; a first flexible covering and sealing film material 20; a wound packing material 26; and, a second flexible covering and sealing material 32; together with a first sealed cavity 22 and a second sealed cavity 34. Thus, many of the basic features of this second embodiment are essentially the same as the first embodiment. However, this second embodiment comprises a port member 50 adhered to the first covering and sealing layer 20. The port member 50 is adapted to receive a multi-lumen conduit 52 which in this case has three lumens 54, 56, 58 therethrough. Two lumens 54, 56 communicate with the first sealed cavity 22 and the third lumen 58 communicates with the second sealed cavity 34. The distal ends of the lumens 54, 56, 58 are in co-operating and operable communication with: vacuum means (not shown) to aspirate the first sealed cavity 22 and to maintain a predetermined vacuum therein; transducer means to monitor and control the pressure within the first sealed cavity; and, pressure/vacuum generating means to apply pressure or vacuum as appropriate to the second sealed cavity, respectively.

The port member 50 will now be described in greater detail. The port member 50 is moulded as a unitary item from a soft plastics material and comprises a body portion 60 having three fluid flow passages 62, 64, 66 therethrough. The inlet/outlet orifices 68, 70, 72 of the three flow passages 62, 64, 66 are contained within a recess 74 having a wall 76 therearound to engage with an outer surface of the co-operating multi-lumen conduit 52 (not shown) as shown in FIG. 2 and sealingly retain within the recess 74. The orifice 72 has a sealing lip 84 therearound to engage with lumen 58 in the end of the conduit 52 for mutual sealing purposes, lumens 54, 56 communicate within the port cavity 74 and are sealed to the port by the outer surface of the triple lumen conduit 52 sealing with the cavity surface 76. The body portion 60 has a flanged face portion 88 extending therearound, the lower face 90 of which is provided with an adhesive layer 92 to enable the port member 50 to be adhered to the first flexible covering and sealing layer 20. Before use the adhesive layer is protected with a siliconised paper layer (not shown) in known manner. When adhered to the first covering and sealing layer 20, the fluid flow passage 64 communicates with a port 96 in the lower face of the port member so as to communicate with the first sealed cavity 22 through an aperture made in the first covering and sealing layer 20 (not shown) and to aspirate fluid inclusive of wound exudate from that cavity and provided a vacuum thereto via the conduit lumen 54. The passage 62 communicates with the first sealed cavity 22 via the port 96, the ends of the passages 62, 64 being both linked to the port 96. However the lumen 56 connected to this flow passage is connected at its distal end to transducer means (not shown) so as to monitor and control the pressure in the first sealed cavity 22. When adhered to the first covering and sealing layer 20 the flow passage 66 communicates with the second sealed cavity via a port 98 thus, the lumen 58 of the conduit 52 is able to apply pressure or vacuum as desired to the second sealed cavity 34. Thus, the port member 50 maintains the first and second sealed cavities independent and sealed from each other.

The port member described above has three fluid flow passages therethrough, however, the port member may has two passages or even more than three passages and may be tailored to provide other functions in additions to those described.

As will be seen from the above two embodiments, it is possible to provide a dressing to both aspirate a wound by TNP therapy giving all of the benefits associated therewith by the first sealed cavity and also simultaneously to work or stress a wound to provide benefits associated with that therapeutic technique in one dressing by pressure pulsing or cycling through positive and negative pressures by the second sealed cavity.

As will be appreciated from the above, complex dressings can be made from component parts held in most hospitals.

In the above embodiments which are illustrative only many variations may be made without departing from the scope of the invention which is limited only by the appendant claims. For example, the sealing drape materials may be in the form of multiple sheets in patchwork form and it is not necessary to rely on self-adhesive films as various adhesives such as stoma adhesive or hydrocolloid may be used to adhere and/or seal the individual layers.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

1. A dressing for the application of topical negative and/or positive pressure therapy to a wound, the dressing comprising:
layer of a pressure resistant porous material adjacent a surface of a wound to be treated;
a first layer of a flexible wound covering and sealing material on top of the optional pressure resistant porous material adapted to surround the wound and seal against sound tissue to form, in use, a first sealed cavity with the wound;
a first conduit having a first end adapted to communicate with an interface between said optional layer of porous material and said first layer of flexible wound covering and sealing material and a second end adapted to communicate with vacuum means to establish a negative pressure between said first covering and sealing material layer and a wound surface;
a resiliency compressible wound packing material on top of said first layer of covering and sealing material;
a second conduit having a first end adjacent said resiliency compressible wound packing material and a second end adapted to communicate with positive or negative pressure generating means; and a second layer of flexible covering and sealing material over said resiliency compressible wound packing to form, in use, a second sealed cavity above said first sealed cavity and said wound.

2. A dressing according to claim 1 wherein the resiliency compressible wound packing material is transparent.

3. A dressing according to claim 1 wherein the resiliency compressible wound packing material is made from a polyurethane material.

4. A dressing according to claim 1 wherein the first layer of flexible covering and sealing material is provided with a port member.

5. A dressing according to claim 4 wherein the port member receives said first and second conduits.

6. A dressing according to claim 5 wherein the first and second conduits are in the form of a single multi-lumen conduit.

7. A dressing according to claim 4 wherein said port member directs fluid flow in said conduits with respect to said first sealed cavity and said second sealed cavity as appropriate.

8. A dressing according to claim 4 wherein said port member is adapted to receive three lumens.

9. A dressing according to claim 8 wherein a third lumen is adapted to be operably connected to transducer means to monitor pressure in said first sealed cavity.

10. A dressing according to claim 4 wherein said port member is bonded to said first layer of flexible covering and sealing material.

11. A dressing according to claim 4 wherein the port member maintains the first and second sealed cavities independent of each other with respect to pressure.

12. A port member for a dressing, the port member comprising a body portion having flow passages adapted to cooperate with at least two lumens; a face portion adapted to be bonded to a flexible membrane material; and, the flow passages being directed on either side of said face portion.

13. A port member according to claim 12 adapted to receive a conduit in the form of a single multi-lumen conduit.

14. A port member according to claim 12 having at least one fluid flow passage configured to communicate with a region on a first side of said face portion.

15. A port member according to claim 12 and having at least one fluid flow passage configured to communicate with a region on a second side of said face portion.

16. A kit for the provision of a topical negative pressure therapy dressing for a wound, the kit comprising: pressure resistant porous material for placement, in use, adjacent a wound surface; flexible covering and sealing material adapted, in use, for adhering to sound skin; resiliently compressible porous wound packing material; a conduit comprising at least two lumens; and, a port member having at least two flow passages adapted to co-operate with said two lumens.

17. A method of providing a dressing including a bag member on a wound on a mammal, the method comprising the steps of:
placing a layer of pressure resistant material which allows for transmission of fluid on a bed of the wound;
placing an end of a first conduit adjacent said optional pressure resistant material; adhering a first layer of a flexible, wound covering and sealing material over the aspirant conduit and pressure resistant material such that said first layer of flexible material is sealed to skin surrounding the wound and to said first conduit so as to form a first sealed cavity over said wound;
placing a resiliently compressible wound packing material in the wound cavity on top of said first sealing layer material; placing an end of a second conduit adjacent said wound packing material; and
adhering a second layer of a flexible, wound covering and sealing material over said wound packing material and an area surrounding said wound to seal thereagainst and to said second conduit so as to form a second sealed cavity over said wound.

18. A method according to claim 17 wherein said first and second flexible, wound covering and sealing materials are semi-permeable materials.

19. A method according to claim 17 wherein the first and second flexible, wound covering and sealing materials are coated with an adhesive.

20. A method according to claim 17 wherein an optional layer of wound packing material is placed on top of the optional layer of pressure resistant wound contact material.

21. A method according to claim 17 wherein the optional layer of pressure resistant material has a surface selected from the group consisting of porous, textured and channelled.

22. A method according to claim 17 wherein the optional layer of pressure resistant material is bio-absorbable.

23. A method according to claim 17 further including the step of applying a negative pressure to the first sealed cavity prior to creating the second sealed cavity.

24. A method according to claim 17 further including the step of including one or more additional conduits in at least one of the first and second sealed cavities.

25. A method according to claim 17 wherein the second sealed cavity is subjected to a pressure range from positive pressures to negative pressures.

26. A method according to claim 17 wherein a fluid is used to apply pressure to said wound by inflating the second cavity.

27. A method according to claim 26 wherein the fluid is temperature controlled.

28. A method according to claim 25 wherein the applied pressure is pulsed.

29. A method according to claim 17 wherein the resiliency compressible wound packing material is a foam created in-situ.
